(19) United States
(12) Patent Application Publication
(10) Pub. No.: US 2011/0046382 A1
(43) Pub. Date: Feb. 24, 2011

(54) PROCESS FOR THE PREPARATION OF 4-[2-(5-ETHYL-2-PYRIDYL)ETHOXY]NITROBENZENE AND PIOGLITAZONE

(75) Inventors: Massimo Ferrari, Cenate Sotto (IT); Marcello Ghezzi, Curno (IT); Emanuele Ghezzi, Montello (IT)

Correspondence Address: GIFFORD, KRASS, SPRINKLE,ANDERSON & CITKOWSKI, P.C, PO BOX 7021, TROY, MI, 48007-7021, US

(73) Assignee: Erregierre S.P.A., San Paolo D'Argon (IT)

(21) Appl. No.: 12/989,877

(22) PCT Filed: Apr. 28, 2008
(86) PCT No.: PCT/IT08/00294

§ 371 Date: Oct. 27, 2010

(51) Int. Cl. C07D 417/12; C07D 213/30

(52) U.S. Cl. 546/269.7; 546/338

(57) ABSTRACT

A process for the preparation of 4-[2-(5-ethyl-2-pyridyl)ethoxy]nitrobenzene is described, which comprises the step of reacting 2-(5-ethyl-2-pyridyl)ethanol with 1-fluoro-4-nitrobenzene in acetone in the presence of an alkali metal hydroxide. The intermediate 4-[2-(5-ethyl-2-pyridyl)ethoxy]nitrobenzene is used for the preparation of pioglitazone.

### FIELD OF THE INVENTION

The present invention relates to a process for the preparation of 4-[2-(5-ethyl-2-pyridyl)ethoxy]nitrobenzene and pioglitazone.

### STATE OF THE ART

The compound 4-[2-(5-ethyl-2-pyridyl)ethoxy]nitrobenzene is a crucial intermediate for the preparation of pioglitazone.

The product pioglitazone, 5-{4-[2-(5-ethyl-2-pyridyl)ethoxy]benzyl}-2-imino-4-thiazolidinedione was disclosed for the first time in document EP0193256 as a member included in a family of compounds characterized by the thiazolidinedione moiety and displaying antidiabetic properties.

To date pioglitazone has been of great importance in the treatment of non-insulin dependent diabetes mellitus (NIDDM) because of its extraordinary properties. The synthesis of the molecule disclosed in patent EP0193256 consists in a first reaction step to obtain the intermediate 4-[2-(5-ethyl-2-pyridyl)ethoxy]nitrobenzene, specifically by reaction of 2-(5-ethyl-2-pyridyl)ethanol with 4-fluoronitrobenzene in dimethyl formamide and tetrahydrofuran in the presence of sodium hydride. The resulting nitro intermediate is converted to the corresponding amino compound through the use of a conventional catalytic reducing system, which leads to a methyl-2-bromo-3-[4-[2-(5-ethyl-2-pyridyl)ethoxy]phenyl]propionate by diazotization in the presence of an aqueous solution of hydrobromic acid and acrylic acid or an ester thereof in the presence of a copper catalyst. The methyl-2-bromo-3-[4-[2-(5-ethyl-2-pyridyl)ethoxy]phenyl]propionate compound is then converted to 5-[4-[2-(5-ethyl-2-pyridyl)ethoxy]benzyl]-2-imino-4-thiazolidinedione by reaction with thiourea in alcohol, and pioglitazone is obtained from the thiazolidinedione compound by hydrolysis with a mineral acid.

A process for the preparation of a class of intermediates of formula:

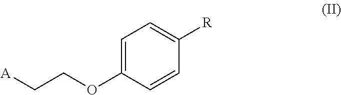

has also been described in document W02007/017095.

This International Application therefore suggests a general process for the preparation of intermediates which may be converted to some thiazolidinedione derivatives, such as rosiglitazone, pioglitazone, troglitazone and ciglitazone, but it only prepares and discloses in detail the intermediate 4-[2-(N-methyl-N-(2-pyridiy)amino)ethoxy]benzaldehyde.

The reaction between a compound of formula

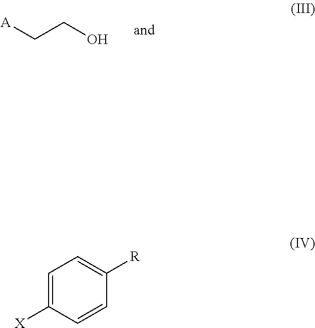

is described in the general process, wherein A may be a pyridyl ring optionally substituted by a C₁-C₄ alkyl group and R may be a nitro group, in the presence of a mixture of a non-polar water-immiscible organic solvent and water, an alkali metal hydroxide or an alkali metal carbonate as a base and a phase transfer catalyst. Toluene is claimed among the solvents and tetrabutyl ammonium bromide is claimed among the phase transfer catalysts.

Document WO03/026586 discloses a novel crystal form of pioglitazone hydrochloride defined as Form II, which has different characteristics from the known Form I of the prior art.

In view of the importance of the pioglitazone compound and of the production of the 4-[2-(5-ethyl-2-pyridyl)ethoxy]nitrobenzene intermediate required to obtain pioglitazone, it is an object of the present invention therefore to provide a process for the preparation of the intermediate 4-[2-(5-ethyl-2-pyridyl)ethoxy]nitrobenzene, which is industrially applicable and advantageous.

It is another object of the invention to provide a process for the preparation of pioglitazone, which avoids also the use of hydrobromic acid, which resulted etching and corrosive in the systems for the preparation of pioglitazone itself.

### SUMMARY

The above mentioned objects have been achieved by a process for the preparation of 4-[2-(5-ethyl-2-pyridyl)ethoxy]nitrobenzene that comprises the step of reacting 2-(5-ethyl-2-pyridyl)ethanol with 1-fluoro-4-nitrobenzene in acetone and in the presence of an alkali metal hydroxide.

The inventors of the present invention have indeed surprisingly found that by using acetone in the reaction between 2-(5-ethyl-2-pyridyl)ethanol and 1-fluoro-4-nitrobenzene the desired intermediate for the preparation of pioglitazone was obtained.

Therefore, in another aspect, the invention relates to a process for the preparation of pioglitazone which comprises the steps of:

a) reacting 2-(5-ethyl-2-pyridyl)ethanol with 1-fluoro-4-nitrobenzene in acetone in the presence of an alkali metal hydroxide;

b) reducing the intermediate 4-[2-(5-ethyl-2-pyridyl)ethoxy]nitrobenzene in 4-[2-(5-ethyl-2-pyridyl)ethoxy]aniline;

c) converting 4-[2-(5-ethyl-2-pyridyl)ethoxy]aniline to methyl-2-chloro-3-[4-[2-(5-ethyl-2-pyridyl)ethoxy]phenyl]propionate in the presence of hydrochloric acid, sodium nitrite and methyl acrylate;

d) transforming methyl-2-chloro-3-[4-[2-(5-ethyl-2-pyridyl)ethoxy]phenyl]propionate in 5-[4[2-(5-ethyl-2-pyridyl)ethoxy]benzyl]-2-imino-4-thiazolidinedione in the presence of thiourea; and

e) hydrolyzing 5-[4-[2-(5-ethyl-2-pyridyl)ethoxy]benzyl]-2-imino-4-thiazolidinedione with hydrochloric acid in order to obtain pioglitazone.

The features and the advantages of the invention will become apparent from the following detailed description.

### DETAILED DESCRIPTION

The invention therefore relates to a process for the preparation of 4-[2-(5-ethyl-2-pyridyl)ethoxy]nitrobenzene that comprises the step of reacting 2-(5-ethyl-2-pyridyl)ethanol with 1-fluoro-4-nitrobenzene in acetone and in the presence of an alkali metal hydroxide.

The alkali metal hydroxide according to the invention may be selected from the group consisting of sodium hydroxide and potassium hydroxide, the alkali metal hydroxide is preferably potassium hydroxide.

The starting material 2-(5-ethyl-2-pyridyl)ethanol is commercially available or may be obtained by means of known organic chemical synthesis.

The weight ratios between 2-(5-ethyl-2-pyridyl)ethanol and 1-fluoro-4-nitrobenzene are preferably in the range from 1:1 to 1:3.

Once the raw product according to the invention is obtained in a solution, it may preferably be extracted by sequential extractions with known water/organic solvent systems, more preferably with water/toluol systems.

The resulting 4-[2-(5-ethyl-2-pyridyl)ethoxy]nitrobenzene compound may therefore be converted to pioglitazone according to the invention.

Specifically, the intermediate 4-[2-(5-ethyl-2-pyridyl)ethoxy]nitrobenzene is mainly reduced to 4-[2-(5-ethyl-2-pyridyl)ethoxy]aniline through palladium-carbon catalyst in the presence of hydrogen, preferably at a pressure in the range from 1 to 4 atm, and the resulting 4-[2-(5-ethyl-2-pyridyl)ethoxy]aniline is then converted to methyl-2-chloro-3-[4-[2-(5-ethyl-2-pyridyl)ethoxy]phenyl]propionate in the presence of hydrochloric acid, sodium nitrite and methyl acrylate. Advantageously, the inventors of the present invention have in fact found that hydrochloric acid could be used instead of the prior art hydrobromic acid, thus obtaining a quantitative yield of methyl-2-chloro-3-[4-[2-(5-ethyl-2-pyridyl)ethoxy]phenyl]propionate, which could then be converted to thiazolidinedione, thus avoiding at the same time the corrosive/etching effect on the systems, which is generally associated to prior art hydrobromic acid.

The resulting methyl-2-chloro-3-[4-[2-(5-ethyl-2-pyridyl)ethoxy]phenyl]propionate compound is converted in the presence of thiourea to 5-[4-[2-(5-ethyl-2-pyridyl)ethoxy]benzyl]-2-imino-4-thiazolidinedione, which is transformed in raw pioglitazone in the presence of hydrochloric acid.

The raw pioglitazone product may then be subjected to purification and subsequently transformed in the corresponding hydrochloride salt through the use of hydrochloric acid in ethanol solvent. The pioglitazone hydrochloride resulting from the process of the invention corresponds to the crystal form defined as Form I in document WO03/026586.

An example of the process of the invention now follows by way of non-limitative example.

### Example 1

### Step a: Preparation of 4-[2-(5-ethyl-2-pyridyl)ethoxy]nitrobenzene

25.0 g of 2-(5-ethyl-2-pyridyl)ethanol and 100 g of acetone were loaded in a flask of appropriate capacity. The mixture was brought to 15-20° C. and 25.0 g of potassium hydrate were added while cooling was maintained. 1-fluoro-4-nitrobenzene (30.0 g) was then poured while maintaining the temperature at 15-20° C. The temperature of 15-20° C. was maintained for at least 4 hours. 20 g of 80% acetic acid, 125 g of distilled water, 75 g of toluol were added after monitoring by TLC, at the end of the reaction and maintaining the temperature in the range of 15-20° C. The solution was subsequently stirred at 15-20° C. for at least 15 minutes, and the lower aqueous phase was then separated and discarded. Distilled water (25 g) was added to the organic phase. After stirring at 15-20° C. for at least 15 minutes, the lower aqueous phase was separated and discarded. 30.0 g of distilled water were then added to the organic phase. The solution was brought to the temperature of 25-35° C., while maintaining in brine, and 32% hydrochloric acid (22.5 g) was poured. The pH was then checked to be lower than 1.0 while maintaining at 30-35° C. for at least 5 minutes. The lower aqueous phase containing the product was separated and the top organic phase was discarded. 20.0 g of toluol were added to the aqueous phase loaded again in the reactor. After stirring at 30-35° C. for at least 5 minutes, the lower aqueous phase containing the product was separated and the top organic phase was discarded. 20.0 g of toluol were added to the aqueous phase loaded again in the reactor and the mixture was stirred at 30-35° C. for at least 5 minutes. The lower aqueous phase containing the product was separated, and the top organic phase was discarded. 60 g of methanol were added to the aqueous phase loaded again in the reactor and the mass was cooled to 10-15° C. 30.0 g of 30% ammonia were poured while maintaining in brine. The mass was maintained at 10-15° C. for at least 30 minutes, then cooled to −10° -0° C. for at least 30 minutes, then centrifuged by washing with 30.0 g of distilled water. The product was dried and 37.0 Kg of corresponding dry product were obtained.

### Example 2

### Step b: Preparation of 4-[2-(5-ethyl-2-pyridyl)ethoxy]aniline

37.0 Kg of 4-[2-(5-ethyl-2-pyridyl)ethoxy]nitrobenzene, 111 Kg of toluol, 1.10 Kg of 5% Pd/C were loaded in a hydrogenator. The mass was heated to 75° C., the hydrogen was then taken to a pressure from 1 to 4 atm until hydrogen was no longer consumed. The catalyst was filtered at the end of the reaction and the toluene solution was used as such in the following step. Approximately 30.0 Kg of product were obtained as determined by titration.

### Example 3

### Step c: Preparation of methyl 2-chloro-3-{4-[2-(5-ethyl-2-pyridyl)ethoxy]phenyl}propionate

30.0 Kg of 4-[2-(5-ethyl-2-pyridyl)ethoxy]aniline in toluene solution were loaded in a reactor and an oily residue was distilled. 30.0 Kg of distilled water and 58 Kg of 32% hydrochloric acid were added to the residue. The mass was cooled to 0° C. and a separately prepared solution of 10.2 Kg of sodium nitrite and 20.4 Kg of distilled water was poured. The resulting solution was poured into another reactor at a temperature of 50° C. containing 90 Kg of acetone, 22.5 Kg of methanol, 1.40 Kg of cuprous oxide, 60 Kg of methyl acrylate.

A solution prepared with 17.7 Kg of potassium carbonate and 35.5 Kg of distilled water was then added. Distillation followed until an internal temperature of 95° C. was reached, then 30.0 Kg of ethyl acetate, 60 Kg of distilled water, 30.0 Kg of 30% ammonia were added. The solution was separated and the lower aqueous phase was discarded. The resulting filtered solution was used in the following step.

### Example 4

### Step d: Preparation of 5-{4-[2-(5-ethyl-2-pyridyl)ethoxy]benzyl}-2-imino-4-thiazolidinedione

The solution of methyl-2-chloro-3-{4-[2-(5-ethyl-2-pyridyl)ethoxy]phenyl}propionate deriving from 30.0 Kg of 4-[2-(5-ethyl-2-pyridyl)ethoxy]aniline in ethyl acetate was loaded in a reactor. Distillation followed until an oily residue was obtained and 10.1 Kg of sodium acetate, 10.1 Kg of thiourea, 1.40 Kg of potassium iodide, 60 Kg of methanol were added. The mass was heated under reflux for 24 hours, then the mass was dry distilled. 90 Kg of ethyl acetate, 90 Kg of distilled water, 9.0 Kg of potassium carbonate were added to the mass. The mass was heated under reflux for 30 minutes, then cooled and centrifuged by washing with 30.0 Kg of distilled water. The resulting wet raw solid was then purified for the treatment with 90 Kg of ethyl acetate, 45 Kg of N,N-dimethyl formamide. The mass was heated to 70° C. and then cooled to 0° C. and filtered by washing with 30.0 Kg of ethyl acetate. After drying approximately 14.0 Kg of the titre product were produced.

### Example 5

### Step e: Preparation of the Raw Base Pioglitazone

14.0 Kg of 5-{4-[2-(5-ethyl-2-pyridyl)ethoxy]benzyl}-2-imino-4-thiazolidinedione, 54 Kg of distilled water, 15.5 Kg 32% hydrochloric acid were loaded in a reactor. The mass was heated to 100° C. and maintained at this temperature for 5 hours. 28.0 Kg of toluol, 14.0 Kg of 30% ammonia were added at the end of the reaction and the mass was then cooled to 0° C. and filtered. Approximately 13.3 Kg of raw base Pioglitazone were obtained.

The pioglitazone product according to the invention was subjected to a purification step and transformed into the corresponding hydrochloride salt.

### Example 6

### Purification step for Raw Base Pioglitazone and transformation into hydrochloride salt

13.3 Kg of raw base pioglitazone and 76 Kg of N,N-dimethyl formamide were loaded in a reactor. The mass was heated to 85° C. in solution, then 39.9 Kg of acetone were added. The mass was cooled to 10° C. and filtered. Approximately 12.6 Kg of base Pioglitazone were obtained.

12.6 Kg of base pioglitazone, 63 Kg of methanol were loaded in a reactor and 1.30 Kg of hydrochloric acid gas were added. The mixture was stirred at 60° C. in solution, then 37.8 Kg of filtered ethanol were added. Approximately 77 Kg of solvent mixture were distilled and the mass was cooled to 55° C. to effective precipitation. The mass was again heated under reflux, then cooled to 0-5° C. for at least 30 minutes. Centrifuging followed by washing with 12.6 Kg of ethanol. The product was dried at 70° C. Approximately 11.5 Kg of Pioglitazone hydrochloride were obtained, in the crystal form defined as Form I in document WO 03/026586.

1. A process for the preparation of 4-[2-(5-ethyl-2-pyridyl)ethoxy]nitrobenzene which comprises the step of reacting 2-(5-ethyl-2-pyridyl)ethanol with 1-fluoro-4-nitrobenzene in acetone in the presence of an alkali metal hydroxide.

2. A process for the preparation of pioglitazone which comprises the steps of:
a) reacting 2-(5-ethyl-2-pyridyl)ethanol with 1-fluoro-4-nitrobenzene in acetone in the presence of an alkali metal hydroxide;
b) reducing the 4-[2-(5-ethyl-2-pyridyl)ethoxy]nitrobenzene intermediate in 4-[2-(5-ethyl-2-pyridyl)ethoxy]aniline;
c) converting 4-[2-(5-ethyl-2-pyridyl)ethoxy]aniline to methyl-2-chloro-3[4-[2-(5-ethyl-2-pyridyl)ethoxy]phenyl]propionate in the presence of hydrochloric acid, sodium nitrite and methyl acrylate;
d) transforming methyl-2-chloro-3-[4-[2-(5-ethyl-2-pyridyl)ethoxy]phenyl]propionate in 5-[4-[2-(5-ethyl-2-pyridyl)ethoxy]benzyl]-2-imino-4-thiazolidinedione in the presence of thiourea, and
e) hydrolyzing 5-[4-[2-(5-ethyl-2-pyridyl)ethoxy]benzyl]-2-imino-4-thiazolidinedione with hydrochloric acid in order to obtain pioglitazone.

3. Process according to claim 1, wherein the alkali metal hydroxide is selected from the group consisting of sodium hydroxide and potassium hydroxide.

4. Process according to claim 3, wherein the alkali metal hydroxide is potassium hydroxide.

5. Process according to claim 1, wherein the weight ratios between 2-(5-ethyl-2-pyridyl)ethanol and 1-fluoro-4-nitrobenzene are preferably in the range from 1:1 to 1:3.

6. Process according to claim 2, wherein in step b) the 4-[2-(5-ethyl-2-pyridyl)ethoxy]nitrobenzene intermediate is reduced to 4-[2-(5-ethyl-2-pyridyl)ethoxy]aniline through a palladium-carbon catalyst in the presence of hydrogen.

7. Process according to claim 2, wherein the raw pioglitazone product is subjected to purification and subsequently transformed into the corresponding hydrochloride salt through the use of hydrochloric acid in ethanol solvent.

8. Process according to claim 2, wherein the alkali metal hydroxide is selected from the group consisting of sodium hydroxide and potassium hydroxide.

9. Process according to claim 2, wherein the weight ratios between 2-(5-ethyl-2-pyridyl)ethanol and 1-fluoro-4-nitrobenzene are preferably in the range from 1:1 to 1:3.
